(19) United States
(12) Patent Application Publication
(10) Pub. No.: US 2024/0324531 A1
(43) Pub. Date: Oct. 3, 2024

(54) METHOD TO INCREASE POLLEN FERTILITY

(71) Applicant: Keygene N.V., Wageningen, (NL)

(72) Inventors: Natalia CARRENO-QUINTERO, Wageningen (NL); Tatyana Mitkova RADOEVA, Wageningen (NL); René Johannes Maria HOFSTEDE, Wageningen (NL)

(21) Appl. No.: 18/737,058

(22) Filed: Jun. 7, 2024

Related U.S. Application Data
(63) Continuation of application No. PCT/EP2022/085101, filed on Dec. 9, 2022.

(30) Foreign Application Priority Data
Dec. 10, 2021 (EP) 21213796.2

(51) Int. Cl. A01H 1/02; A01G 7/00; A01H 5/10

(52) U.S. Cl. CPC A01H 1/027; A01G 7/00; A01H 5/10

(57) ABSTRACT

The invention pertains to a method for increasing plant pollen viability using a MRN-ATM pathway inhibitor. The increased pollen viability preferably results in increased plant viability. The invention further pertains to an MRN-ATM pathway inhibitor for increasing plant pollen viability. A preferred MRN-ATM pathway inhibitor for use in the invention is 2-Amino-5-[(4-hydroxyphenyl)methylene]-4(5H)-thiazolone. The invention also pertains to a method for developing a mature fertile plant graft comprising contacting an isolated plant part comprising an immature flower bud with a (hazardous or toxic) compound.

### CROSS-REFERENCE TO RELATED APPLICATION

This application is a continuation of International PCT Application PCT/EP2022/085101 filed Dec. 19, 2022, which application claims priority to European Patent Application No. 21213796.2, filed Dec. 10, 2021, the entire contents of which are hereby incorporated by reference herein.

### FIELD OF THE INVENTION

The present invention is in the field of plant breeding, more particular the field of interspecific hybrid plant breeding. Disclosed are new methods to improve plant fertility via treatment of pollen resulting in increased pollen viability and the use of a compound to increase pollen fertility. Said pollen may be from a low or infertile plant such as an interspecific hybrid.

### BACKGROUND

The process of plant breeding is highly dependent on the availability of genetic variation. This variation can spawn from reverse breeding technologies, CRISPR/Cas9 and by using natural variation found in closely related wild species of a crop of interest. It is desired to introgress novel traits from wild relatives for different reasons, e.g. to increase pest resistance of a cultivar and/or to create novel varieties. Moreover, hybrid species often exhibit heterosis or hybrid vigor, which in general increases the biological quality. Hybrid species are thus of great interest and interspecific hybridization is expected to stay a powerful tool for breeding in the coming decades.

Several reproductive barriers exist for the creation and propagation of interspecific hybrids, depending on the species that are being crossed. These post-and prezygotic barriers that keep different plant species from crossing range from problems with the pollen-stigma interaction, pairing and recombination between the homeologous chromosomes during meiosis in the F₁ hybrid, and pollen tube guidance to embryo or endosperm development. Current methods to circumvent these problems include bud pollination, ovule cultures and embryo rescue, which are still far from optimal. So far the creation of interspecific hybrids is greatly dependent on trial and error, involving large scale breeding efforts, which require large quantities of time, effort and costs. Even more challenging may be the subsequent propagation of these obtained interspecific hybrids. Indeed, the fast majority of interspecific hybrids do not have viable pollen.

Therefore, there is a strong need in the art for a method that improves plant fertility, especially for plants with low fertility such as interspecific hybrids. More in particular there is a strong need for increasing pollen viability, for instance in interspecific hybrid breeding and/or production.

### SUMMARY

The invention may be summarized in the following embodiments:

Embodiment 1. A method for improving the viability of plant pollen, wherein the method comprises the steps of:
a) providing a first seed plant or plant part thereof comprising a tissue giving rise to plant pollen;
b) inhibiting the MRN-ATM pathway in at least part of the provided plant or plant part; and
c) allowing the plant or plant part to produce pollen, wherein the pollen shows improved viability.

Embodiment 2. The method according to embodiment 1, wherein the MRN-ATM pathway is inhibited in step b) by contacting said plant or plant part to 2-Amino-5-[(4-hydroxyphenyl)methylene]-4(5H)-thiazolone.

Embodiment 3. The method according to embodiment 1 or 2, wherein the improved viability of plant pollen increases plant fertility.

Embodiment 4. The method according to any one of the preceding embodiments, wherein the first plant or plant part thereof of step a) is an interspecific hybrid or part thereof.

Embodiment 5. The method of any one of the preceding embodiments, wherein the plant part provided in step a) is a plant cutting, and wherein the cutting edge of the plant cutting is contacted in step b) with the compound that inhibits the MRN-ATM pathway.

Embodiment 6. The method according to embodiment 5, wherein the plant cutting is a cutting of an angiosperm seed plant and comprises an inflorescence, wherein said inflorescence comprises at least one immature flower bud and preferably does not comprise a mature flower bud.

Embodiment 7. The method according to any one of the preceding embodiments, wherein steps a)-c) are performed using a plant part that is a scion, and wherein the method further comprises the step of:
d) grafting the scion onto a stock.

Embodiment 8. Method of any one of the preceding embodiments, wherein the method further comprises the step of allowing the pollen to mature.

Embodiment 9. The method according to embodiment 8, wherein the method further comprises the step of isolating the mature pollen.

Embodiment 10. The method according to any one of the preceding embodiments, wherein the method further comprises the step of self-pollinating the first plant or pollinating a second plant. Embodiment 11. Plant growth medium comprising a compound inhibiting the MRN-ATM pathway.

Embodiment 12. Viable pollen of a plant or plant part obtainable by a method of any one of embodiments 1-10.

Embodiment 13. A method for producing a first seed plant having improved pollen viability, wherein the method comprises the steps of:
a) providing a first seed plant or plant part thereof comprising a tissue giving rise to plant pollen;
b) inhibiting the MRN-ATM pathway in at least part of the provided plant or plant part; and
c) allowing the plant or plant part to produce pollen, wherein the pollen shows improved viability.

Embodiment 14. A seed plant, preferably an interspecific hybrid, comprising pollen having an improved viability obtainable by the method of embodiment 13.

Embodiment 15. Use of a compound inhibiting the MRN-ATM pathway, optionally comprised in a plant growth medium, for improving pollen viability.

Embodiment 16. Use of the viable pollen of embodiment 12 or the seed plant of embodiment 14 for the pollination of a second plant, preferably for the production of a progeny plant.

### DEFINITIONS

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

Methods of carrying out the conventional techniques used in methods of the invention will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al. Molecular Cloning. A Laboratory Manual, 2nd Edition, Cold Spring Harbor Laboratory Press, Cold Spring Harbor, N. Y., 1989; Ausubel et al.. Current Protocols in Molecular Biology, John Wiley & Sons, New York, 1987 and periodic updates; and the series Methods in Enzymology, Academic Press, San Diego.

“A,” “an,” and “the”: these singular form terms include plural referents unless the content clearly dictates otherwise. Thus, for example, reference to “a cell” includes a combination of two or more cells, and the like.

As used herein, the term “about” is used to describe and account for small variations. For example, the term can refer to less than or equal to ±10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

“And/or”: the term “and/or” refers to a situation wherein one or more of the stated cases may occur, alone or in combination with at least one of the stated cases, up to with all of the stated cases.

“Plant”: is to be understood herein as a plant comprising at least part of a root system and part of a shoot system. The plant preferably comprises a plant part a defined herein below. Preferably, the plant comprises at least a tissue that gives rise to plant pollen. Preferably, the plant comprises at least one of a (mature or immature) flower bud and an inflorescence. Non-limiting examples of plants include crop plants and cultivated plants, such as barley, cassava, cotton, groundnuts or peanuts, maize, millet, oil palm fruit, potatoes, pulses, rapeseed or canola, rice, rye, sorghum, soybeans, sugar cane, sugar beets, sunflower, wheat, okra, allium, celery, asparagus, wax gourd, beet, Brassica including vegetable Brassica , bell pepper, hot pepper, endive, chicory, melon including watermelon, cucumber, gherkin, zucchini, pumpkin, artichoke, carrot, rocket, fennel, lettuce, bottle gourd, ridge gourd, sponge gourd, bitter gourd, parsnip, parsley, common bean, bladder cherry, tomatillo, radish, eggplant, tomato including tomato rootstock, pepino, spinach, snake gourd, corn salad, pea including chick pea, soybean, broad bean, sweet corn, hemp, hop, berries, dandelion and ornamentals.

“Plant part”: this includes at least one of plant cells, plant protoplasts, plant cell tissue cultures from which plants can be regenerated, plant calli, plant clumps, (mature or immature) flower bud, inflorescence, and plant cells that are intact in plants or parts of plants such as embryos, pollen, ovules, seeds, leaves, flowers, branches, fruit, kernels, ears, cobs, husks, stalks, roots, root tips, anthers, grains and the like. Preferably, the plant part comprises at least a tissue that gives rise to plant pollen. Preferably, the plant part is at least one of a (mature or immature) flower bud and an inflorescence.

A “seed plant” as described herein is a plant or group of plants that bears seeds, as the main from of sexual reproduction, as opposed to spore forming plants. The group is composed of gymnosperms and angiosperms, otherwise known as spermatophytes, and encompasses all agro-cultural relevant crops. Non-limiting examples are herein provided under the definition of “plant”.

An “inflorescence” is a reproductive unit of a plant. It is a preferably a group or cluster of flowers arranged on a stem, that is composed of a main branch or an arrangement of branches. It can be the part of the shoot of seed plants where the flowers are formed. Two specific types of inflorescence can be determined, a determinate and an indeterminate inflorescence. A determinate (cymose) inflorescence grows a terminal (central) flower from a terminal bud, with arrest of the elongation of the central axis. In an indeterminate (racemose) inflorescence the terminal bud keeps growing and forming lateral flowers from a lateral bud(s). A terminal flower is never formed. Inflorescence is instrumental to the sexual reproduction of said plant. As such it includes, but is not limited to one or more, preferably all, developmental stages of the carpels, anthers, stigma, style, stamen, petals and sepals.

An “interspecific hybrid” is a direct offspring (“F1” offspring) from a cross of two individuals from two species, preferably from within the same genus. The offspring preferably displays traits and characteristics of both parents, but are often sterile, preventing gene flow between the species (Keeton, William T. 1980. Biological science. New York: Norton. ISBN 0-393-95021-2, p. 800). Sterility is often attributed to the different number of chromosomes between the two species. Sterility may be, at least partly, due to the absence of viable pollen.

An “MRN-ATM pathway” is a pathway involved in the control of DNA damage, more specifically in double-strand (DSB) break repair. The MRN-ATM pathway is highly conserved between plant species (Yoshiyama et al., Biology (Basel). 2013 2(4): 1338-1356; Amiard et al. The Plant Cell, 2010, 22 (9): 3020-3033). The MRN complex in plants is a heterotrimer comprising the three proteins Meiotic recombination 11 (Mre11), DNA repair protein Rad50 and Nijmegen breakage syndrome 1 (Nbs1). It is known as the major catalytic protein complex in coordinating and sensing DSBs and in initiating the DNA damage response pathway. The MRN complex senses DSBs and recruits the inactive dimer ATM (ataxia telangiectasia mutated) where it is activated to phosphorylate members of the MRN complex, in addition to a variety of other proteins involved in cell-cycle control and DNA repair (Kanaar and Wyman Cell 2008, 135, 14-16.) Activation of the MRN-ATM pathway results in the activation of DNA damage repair and continuation of the cell-cycle. During meiosis in plants, the subunits of the MRN-ATM pathway are involved in the regulation of meiosis and pollen formation (Culligan et al., Plant J. 2008 August; 55(4): 629-38.; Kurzbauer et al., The Plant Cell 2021; 33(5): 1-24).

An “MRN-ATM pathway inhibitor” is a compound that downregulates or prevents activation of the MRN-ATM pathway. Said compound preferably is a chemical compound.

The term “pollination” or “pollinating” as used herein refers to the process by which pollen is transferred from the anther (male part) to the stigma (female part) of the plant, thereby enabling fertilization and reproduction. Each pollen grain is a male haploid gametophyte, adapted to being transported to the female gametophyte, where it can effect fertilization by producing the male gamete (or gametes), in the process of double fertilization.

A successful angiosperm pollen grain (gametophyte) containing the male gametes is transported to the stigma, where it germinates and its pollen tube grows down the style to the ovary. Its two gametes travel down the tube to where the gametophyte(s) containing the female gametes are held within the carpel. One nucleus fuses with the polar bodies to produce the endosperm tissues, and the other with the ovule to produce the embryo. Gymnosperm lack fruits and flowers. However similarly to angiosperms, gymnosperms use pollen to facilitate fertilization.

### DETAILED DESCRIPTION

The inventors discovered that sterile interspecific hybrids can develop fully matured anthers and viable pollen upon inhibition of the MRN-ATM pathway in a plant.

Therefore in a first aspect of the invention, provided is a method for improving the viability of plant pollen, wherein the method comprises the steps of:
a) providing a first seed plant or plant part thereof comprising a tissue giving rise to plant pollen;
b) inhibiting the MRN-ATM pathway in at least part of the provided plant or part; and
c) allowing the plant or plant part to produce pollen, wherein the pollen shows improved viability.

The method of the invention may also be considered a method for producing plant pollen having improved viability.

### Step a) Providing a First Seed Plant or Plant Part

In step a) of the method of the invention a first seed plant or plant part is provided. Preferably, the provided seed plant comprises a tissue giving rise to plant pollen. Equally, preferably the provided plant part comprises a tissue giving rise to plant pollen. The provided seed plant preferably is an agro-cultural relevant crop plant. It is understood herein that the plant part is a part of the first seed plant.

The term “a tissue giving rise to plant pollen” is well-known to the person skilled in the art. Such tissue preferably is a sporogenous tissue, preferably comprising at least one of sporogenous cells, microsporocytes (also known as “pollen mother cells” or “meiocytes”) and microspores. The tissue may be part of, or comprised within, an immature bud.

Preferably, the first seed plant or plant part thereof provided in step a) comprises an immature bud. Preferably the plant part is a plant cutting. Hence preferably the plant cutting comprises an immature bud.

Preferably, the tissue giving rise to plant pollen may be part of, or comprised within, an inflorescence. Preferably, the plant part provided in step a) is or comprises an inflorescence. Preferably, the plant cutting is or comprises an inflorescence. The inflorescence preferably comprises at least one immature flower bud. The inflorescence may comprise at least 1, 2, 3, 4, 5, 5, 6, 7, 8, 9, 10 or more immature flower buds. Preferably, the inflorescence comprises less than 10, 9, 8, 7, 6, 5, 4, 3, 2, or less than 1 mature flower buds. Preferably the plant part for use in the method of the invention is an inflorescence comprising at least one immature flower bud and does not comprise any mature flower buds. Optionally, any mature flower buds have been removed from the plant or plant part prior to step a) of the method of the invention.

Preferably, the provided first seed plant or part thereof is an angiosperm seed plant. Preferably, the plant cutting is a cutting of an angiosperm seed plant.

The first seed plant preferably has a reduced capacity to produce viable pollen. Preferably, at least about 2%, 4%, 6%, 8%, 10%, 15%, 20%, 25%, 30%, 35%, 40%, 45%, 50%, 55%, 60%, 65%, 70%, 75%, 80%, 85%, 90%, 95% or 100% of the pollen produced by the first seed plant is inviable. Preferably, less than about 95%, 90%, 85%, 80%, 75%, 70%, 65%, 60%, 55%, 50%, 45%, 40%, 35%, 30%, 25%, 20%, 15%, 10%, 8%, 6%, 4%, 2% or less than about 1% of the pollen produced by the first seed plant is viable. Preferably, the first seed plant is unable to produce viable pollen, the first seed plant may be unable to produce any viable pollen. The reduced capacity of the first seed plant to produce viable pollen preferably results in a plant that has a limited ability or is not able to produce offspring through pollination.

The viability of the plant pollen can be straightforwardly determined using any conventional method known to the skilled person. As a non-limiting example, viability can be determined using a conventional MTS (3-(4,5-dimethylthiazol-2-yl)-5-(3-carboxymethoxyphenyl)-2-(4-sulfophenyl)-2H-tetrazolium) or MTT (3-(4,5-dimethylthiazol-2-yl)-2,5-diphenyltetrazolium bromide) assay, for example using the method steps as described herein below in example 1.

Alternatively or in addition, preferably in the provided first seed plant, at least about 2%, 4%, 6%, 8%, 10%, 15%, 20%, 25%, 30%, 35%, 40%, 45%, 50%, 55%, 60%, 65%, 70%, 75%, 80%, 85%, 90%, 95% or 100% of the anthers remain closed upon pollen maturation. Preferably, less than about 95%, 90%, 85%, 80%, 75%, 70%, 65%, 60%, 55%, 50%, 45%, 40%, 35%, 30%, 25%, 20%, 15%, 10%, 8%, 6%, 4%, 2% or less than about 1% of the anthers open upon pollen maturation. Opening of anthers can be assessed by visual inspection, optionally using a microscope for magnification of the image.

The first seed plant used in the method of the invention may be a plant with a low pollen viability or non-viable pollen and/or a plant with closed anthers and/or a plant with low fertility. Optionally the plant is sterile or partly sterile. The first seed plant may be an interspecific hybrid. A first seed plant is understood herein to be a plant that produces seeds as the main form of sexual reproduction and is a member of the class of the Spermatophytes and contains but is not limited to all mayor crops. The first seed plant provided in step a) may be a angiosperm or a gymnosperm preferably an angiosperm. Preferably in step a) a plant part of an angiosperm is provided, preferably an inflorescence from an angiosperm. The plant may belong to, but is not limited to, a plant of the family of Solanaceae, Cucurbitaceae, Brassicaceae, Asteraceae, Malvaceae, Fabaceae, Poaceae, Amaranthaceae, Leguminosac, Musaccac, Gramineae, Rosaceae, Euphorbiaceae, Apiaceae and Rubiaceae. The plant may belong to, but is not limited to, a plant of the genus Solanum (including Lycopersicum), Allium, Beta, Brassica, Nicotiana, Capsicum, Petunia, Triticum, Oryza, Glycine, Cucumis, Cichorium, Chrysanthemum, Pennisetum, Secale, Lactuca, Hordeum, Helianthus, Cannabis, Avena, Sorghum, Gossypium, Medicago Phaseolus, Sorghum, Saccharum, Hordeum, Taraxacum, Pisum, Zea, Medicago, Phaseolus, Rosa, Lilium, Coffea, Linum, Mannihot, Daucus, Cucurbita, Citrullusand Musa. Preferably the plant is a plant of the family Brassicaceae, preferably a plant of the genus Brassica.

The provided first seed plant may be a monoecious or a dioecious plant. Preferably the provided first seed plant is a plant with a reduced or low fertility, or a plant that is infertile. Preferably the first seed plant is a plant with a reduced or low pollen viability, or a plant that has non-viable pollen. Preferably, the first seed plant has a limited ability or is not able to produce offspring through pollination.

The first seed plant may be an interspecific hybrid. The interspecific hybrid may result from a cross between any combination of species that can result in viable offspring. In other words, the interspecific hybrid may be a hybrid of any two parent plants of a different species that result in a viable plant. Preferably, the interspecific hybrid is a hybrid between two species of the same genus. This interspecific hybrid may be characterized by hybrid vigour over the parent plants and/or having a mix of chromosomes from each parent.

Preferably, the interspecific hybrid is a hybrid between two parent plants that differ in number of chromosomes and/or in ploidy. The invention is not limited any particular interspecific hybrid but is understood to be generally applicable. The method of the invention may be applied to any interspecific hybrid, i.e. a plant obtainable from any cross between two species, preferably of the same genus, that result in an party or completely sterile interspecific hybrid and/or that could benefit from a method to increase pollen viability, a method of opening anthers, and/or a method to increase fertility.

Optionally, the interspecific hybrid is an interspecific hybrid between two species of the genus Brassica, wherein preferably the parents differ in chromosome number. The interspecific hybrid may be a hybrid of one parent plant being of a species selected from the group consisting of B. rapa (AA 2n=20), B. nigra (BB 2n=16) and B. oleracea (CC 2n=18) and another parent plant being of another species of said group. The interspecific hybrid may be an interspecific hybrid of a cross of Brassica oleraceae with Brassica rapa. Optionally, the interspecific hybrid is a hybrid between two species of the genus Lactuca. The interspecific hybrid may be an interspecific hybrid of a cross of Lactuca sativa with a Lactuca virosa.

Optionally, the first seed plant is an offspring of an interspecific hybrid, preferably an offspring having reduced pollen viability. Said offspring may be an offspring resulting from back-crossing, selfing, or a combination thereof, of an interspecific hybrid. Optionally, in case the interspecific hybrid is considered as a “first generation plant”, said offspring is of the second, third, fourth, fifth, sixth, seventh, eighth, ninth or tenth generation.

### Step b) Inhibiting the MRN-ATM Pathway in the Provided Plant or Plant Part

In step b) of the method of the invention, the MRN-ATM pathway may be inhibited in at least part of the plant. Preferably, the MRN-ATM pathway is at least inhibited in a tissue giving rise to plant pollen. Inhibition of the MRN-ATM pathway may encompass the inhibition of the activity of a protein that is a member of said pathway, wherein preferably the protein is selected from the group consisting of the proteins Mre11, Rad50, Nbs1 (together the MRN complex) and ATM. Preferably inhibition of the MRN-ATM pathway may encompass the inhibition of the MRN complex, preferably the inhibition of the activity of a protein that is a member of said complex, e.g. an inhibition of the activity of at least one of Mre11, Rad50 and Nbs1.

A reduction of the activity of Mre11 is preferably a reduction of at least 10%, 20%, 30%, 40%, 50%, 60%, 70%, 80%, 90% or 100% reduction of activity. A reduction of the activity of Rad50 is preferably a reduction of at least 10%, 20%, 30%, 40%, 50%, 60%, 70%, 80%, 90% or 100% reduction of activity. A reduction of the activity of Nbs1 is preferably a reduction of at least 10%, 20%, 30%, 40%, 50%, 60%, 70%, 80%, 90% or 100% reduction of activity. A reduction of the activity of ATM is preferably a reduction of at least 10%, 20%, 30%, 40%, 50%, 60%, 70%, 80%, 90% or 100% reduction of activity. A 100% reduction of activity is understood herein as the complete absence of activity.

Inhibition of the MRN-ATM pathway may be partial or complete inhibition. Inhibition of the MRN-ATM pathway is preferably performed by exposure to a compound. Inhibiting the MRN-ATM pathway in a tissue giving rise to plant pollen of the first seed plant or part thereof of step b) may then also be regarded as treating a tissue giving rise to plant pollen of the first seed plant, or part thereof, with an MRN-ATM inhibitor. The tissue giving rise to plant pollen is preferably part of, or comprised within, an inflorescence. Inhibiting the MRN-ATM pathway in an inflorescence of the first seed plant or part thereof of step b) may then also be regarded as treating an inflorescence of said first seed plant or part thereof with an MRN-ATM inhibitor.

In addition or alternatively, the MRN-ATM pathway may be inhibited or blocked by downregulating or abolishing the expression of one or more proteins of said pathway, for instance by interfering with the expression of said one or more proteins for instance by silencing using RNAi and/or by mutation of the gene encoding said one or more protein. Mutation of a gene encoding said one or more proteins may result in decreased or abolished expression of a functional protein. Said mutation could be in a promoter sequence resulting in reducing and/or abolished expression and/or in the coding sequence resulting in a dysfunctional and/or truncated protein and/or a protein with a reduced function. Optionally, inhibition of the MRN-ATM pathway is by RNAi, wherein the expressed siRNA/miRNA is under control of a promoter, preferably a tissue-specific promoter. The tissue-specific promoter is preferably active, optionally only active, in the tissue giving rise to plant pollen.

Preferably, the MRN-ATM pathway is inhibited by exposure to a compound. Therefore, step b) of the method of invention may also be step of contacting at least part of the provided plant or plant part to a compound inhibiting the MRN-ATM pathway. Preferably, at least the tissue giving rise to the plant pollen is contacted with said compound. Preferably said compound is a chemical compound, wherein said compound preferably inhibits the 3′-to 5′ exonuclease activity associated with Mre11. Said compound may be 2-Amino-5-[(4-hydroxyphenyl)methylene]-4(5H)-thiazolone, having molecular formula C₁₀H₈N₂O₂S and is also known under the name mirin (PubChem identifier 1206243, CAS Number 299953-00-7). Optionally, the compound for use in the method of the invention is a derivative or an analogue of Mirin. A preferred derivative of Mirin is a derivative as described in Shibata et al., Mol Cell (2014) 53:7-18, which is incorporated herein by reference.

Other potentially useful inhibitory compounds may be inhibitors of ATM kinase activity, such as, but not limited to, any one of:
2-((2S,6R)-2,6-Dimethylmorpholino)-N-(5-(6-morpholino-4-oxo-4H-pyran-2-yl)-9H-thioxanthen-2-yl)acetamide having molecular formula C₃₀H₃₃N₃O₅S and is also known under the name KU-60019 (PubChem identifier 15953870, CAS Number 925701-49-1);
2-(4-Morpholinyl)-6-(1-thianthrenyl)-4H-pyran-4-one having molecular formula C₂₁H₁₇NO₃S₂ and is also known under the name KU-55933 (PubChem identifier 5278396, CAS Number, 587871-26-9); and
1-(6,7-Dimethoxyquinazolin-4-yl)-3-(pyridin-2-yl)-1H-1,2,4-triazol-5-amine having molecular formula C₁₇H₁₅NO₂ and is also known under the name CP-466722 (PubChem identifier 44551660, CAS number 1080622-86-1).

Inhibiting the MRN-ATM pathway is preferably performed by inhibiting Mre11. Preferably, the 3′ to 5′ exonuclease activity associated with Mre11 is inhibited. Preferably said Mre11 inhibitor is mirin.

The whole provided plant may be contacted to a compound that inhibits the MRN-ATM pathway, also indicated herein as “the inhibitory compound”. Optionally, only a part of the plant is contacted with the inhibitory compound. As a non-limiting example, only (part of) the root system or the shoot system is contacted with the inhibitory compound. Optionally only the part of the plant is contacted with the inhibitory compound, but the compound subsequently travels through the plant tissue and may reach the tissue giving rise to the pollen and/or the cells of the immature flower buds.

The MRN-ATM pathway of all cells of the provided plant or plant part may be inhibited. Alternatively, the MRN-ATM pathway is inhibited in only a portion of the cells. For example, the MRN-ATM pathway may be inhibited in at least about 0.5%, 1%, 2%, 5%, 10%, 20%, 30%, 40%, 50%, 60%, 70%, 80%, 90%, 95% or 100% of the plant cells of the provided plant or plant part. The

MRN-ATM pathway may be inhibited in cells of the root system or cells of the shoot system, preferably in cells of the shoot system. The MRN-ATM pathway is preferably inhibited at least in the tissue giving rise to plant pollen. The MRN-ATM pathway may be inhibited at least in one or more immature flower buds. The MRN-ATM pathway may be inhibited at least in a florescence of the provided plant or plant part.

Preferably, at least a plant part is contacted with the inhibitory compound. The MRN-ATM pathway may be inhibited in step b) by contacting at least the tissue giving rise to plant pollen to the inhibitory compound. Preferably, the compound only contacts said tissue giving rise to plant pollen, or contacts only an immature flower bud, or contacts only an inflorescence or contacts only the plant part provided in step a). Preferably, at least a tissue giving rise to plant pollen is contacted with a compound that inhibits the MRN-ATM pathway. Preferably, at least one or more immature flower buds of the provided plant or plant part are contacted with the inhibitory compound. Preferably, at least an inflorescence of the provided plant or plant part is contacted with the inhibitory compound.

The chemical compound may be added by any suitable means known in the art, such as, but not limited to, spraying the flower bud and/or the foliage with a composition comprising the chemical compound, dipping the flower buds in a medium comprising the chemical compound, and admixing the compound to the soil where the plant is growing in order to for the compound to reach the flower buds via its roots.

The MRN-ATM inhibitor may be a compound that is toxic for humans in the concentration required for the method of the invention. Therefore preferably, the compound is not used in an aerosol form and preferably the amount of chemical compound to be used is minimized.

In a preferred embodiment, the plant part comprising a tissue giving rise to plant pollen may be cut off from the remainder of the plant. The plant part may be a plant cutting. Preferably, the plant part may be placed with its cutting edge in growth medium comprising said chemical compound, preferably in a minimal amount of growth medium. In order to prevent the compound to evaporate into the environment, the compound-comprising growth medium can be covered or sealed off, e.g. with a parafilm. Optionally, the compound may be applied as shown in FIG. 1.

Preferably, the MRN-ATM pathway inhibitor is only used on a specific plant part. Preferably, the MRN-ATM pathway is inhibited in a tissue giving rise to plant pollen of said plant part. Said tissue is preferably part of, or comprised within, a flower bud. The plant part therefore comprises preferably at least one flower bud. Preferably, said flower buds are immature and the MRN-ATM pathway is inhibited throughout the period of bud maturation to an open flower. Preferably, said flower buds are immature and the plant or plant part is exposed to a compound inhibiting the MRN-ATM pathway throughout the period of bud maturation to an open flower. Preferably, said MRN-ATM pathway is inhibited temporarily between the moment of development of an immature flower bud to the moment the bud has matured to an open flower. Preferably, the plant or plant part is exposed to a compound inhibiting the MRN-ATM pathway temporarily between the moment of development of an immature flower bud to the moment the bud has matured to an open flower. Preferably, in step a) of the method of the invention, provided is an inflorescence comprising at least one immature flower bud. Preferably, said inflorescence does not comprise mature flower buds. Preferably, said immature flower buds have not yet formed pollen. Said inflorescence may be obtained by removing mature flower buds and only maintaining one or more immature flower buds.

Preferably prior to contacting first seed plant or plant part thereof to the MRN-ATM pathway inhibitor, a part of the plant comprising at least one immature flower bud is cut off from the first seed plant. Preferably in step b) an inflorescence comprising at least one immature flower bud is a cut off from a plant. Preferably subsequently, the part of the plant comprising the immature flower bud is placed with its cutting edge into medium comprising the MRN-ATM pathway inhibitor in a concentration that is effective to inhibit the MRN-ATM pathway, preferably the MRN complex within said pathway, even more preferably the concentration is effective to inhibit Mre11, preferably within the tissue and/or cells giving rise to plant pollen. Preferably said medium is a medium that allows the immature bud(s) to mature. Such medium may be Murashige skoog including vitamins as described in Murashige T. and Skoog F., Physiol. Plant, 15, 473 (1962).

The concentration of the MRN-ATM pathway inhibitor of step b) is present in an effective concentration. An effective concentration is to be understood herein as concentration of the inhibitor that is sufficient to elicit the desired effect, i.e. resulting in increased pollen viability and/or opening of anthers. In case the MRN-ATM pathway inhibitor is mirin, said concentration may be a concentration up to about 5 μM, 4 μM, 3 μM, 2 μM, 1 vM, 900 nM, 800 nM, 700 nM, 600nM, 500 nM, 400 nM, 300 nM, 200 nM, 100 nM, 90 nM, 80 mM, 70 nM, 60 nM, 50 nM, 40 nM or 30 nM, and may be a concentration of at least about 1nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM or 20 nM. Preferably said concentration is in the range of between about 1 nM and 5 μM, between about 1 nM and 1 μM, between about 5 nM and 500 nM, between about 5 nM and 100 nM, between about 10 nM and 100 nM, between about 5 nM and 50 nM, between about 10 nM and 50 nM, or preferably between about 20 nM and 30 nM.

Preferably an immature flower bud of the first seed plant or plant part thereof provided in step a) of the method of the invention is treated with MRN-ATM pathway inhibitor in step b) for the duration of maturation of said flower bud, preferably in a medium comprising said inhibitor in a concentration as indicated herein. The treatment can take effect on the immature flower bud before pollen formation and may end when pollen is formed. Preferably, the flower bud is considered mature if it is opened. Preferably the flower bud is treated with MRN-ATM pathway inhibitor by placing a inflorescence comprising said flower bud in a medium comprising the MRN-ATM pathway inhibitor in an effective concentration as indicated herein during maturation of said flower bud, i.e. starting when the flower bud is immature up to the time when the flower bud is opened.

Preferably the tissue giving rise to plant pollen, preferably an immature flower bud, is treated with an MRN-ATM pathway inhibitor for at least about 1 hour, 2 hours, 4 hours, 6 hours, 8 hours, 16 hours, 1 day, 2 days, 3 days, 4 days, 5 days, 6 days, 7 days, 8 days, 9 days or 10 days. Preferably, the flower bud is treated up to about 20 days, 19 days, 18 days, 17 days, 16 days, 15 days, 14 days, 13 days, 12 days, 11 days, 10 days, 9 days, 8 days, 7 days, 6 days, 5 days, 4 days, 3 days, 2 days or 1 day. Preferably, the tissue giving rise to plant pollen, preferably the immature flower bud, is treated for a period between about 1 hour and 20 days, between about 1 day and 20 days, between about 1 day and 10 days, between about 5 days and 15 days or between about 8 days and 12 days.

The treatment period may be dependent on the duration of the maturation period of the flower bud. The period for maturation, may be known or determined by the person skilled in the art. Optionally, the immature flower bud is only treated during part of the maturation period. The period for maturation of the flower bud preferably includes at least microsporogenesis involving the pre-meiotic phase, the first meiotic division and optionally the second meiotic division. Hence the period for treating the immature flower bud with the compound of the method of the invention is preferably during at least microsporogenesis involving the pre-meiotic phase and the first meiotic division, and optionally also during the second meiotic division.

The treatment may be during the complete maturation period of the flower bud, e.g. 100% of the time, or a part of said period, e.g. about 20%, 40%, 60%, 80%, 90%, 95%, 96%, 97%, 98%, 99% of the time period required for the maturation of the immature flower bud.

### Step c) Production of Pollen Having an Improved Viability

Improved pollen viability is to be understood herein as the increased pollen viability of a first seed plant or part thereof hat has been treated by the method of the invention, as compared to the pollen of a control plant or plant part thereof.

Preferably, the improved pollen viability may be determined by comparing the pollen viability of a tissue giving rise to plant pollen and that has been treated by the method of the invention, as compared to tissue giving rise to plant pollen that has not been treated by the method of the invention, also indicated herein as a control tissue. Preferably the treated tissue and the control tissue are of the same species, preferably of the same plant. Preferably, the treated tissue and the control tissue have a similar or the same genetic background.

Preferably, the improved pollen viability may be determined by comparing the pollen viability of a flower bud that has been treated by the method of the invention, as compared to the flower bud of a control plant or plant part thereof, also indicated herein as a control flower bud.

The control flower bud is a flower bud that is comparable or identical to the flower bud of the method of the invention and only differs in that it has not undergone the method of the invention. In case the method of the invention comprises treating a plant part comprising an immature flower bud, a similar plant part may serve as a control. Said similar plant part may be of the same or a similar plant and preferably has underwent the same treatment as the plant part treated with the method of the invention, with the exception that said similar plant part was not contacted with an MRN-ATM pathway inhibitor as described herein. Preferably, said control plant or plant part comprising a (control) flower bud of the same developmental stage is placed in the same medium for the same period of time as the plant or plant part of the method of the invention albeit without an MRN-ATM pathway inhibitor, i.e. the concentration of the MRN-ATM pathway inhibitor being zero. A similar plant or plant part may be a plant or a plant part of the same species or being an offspring of the same parent plants. Preferably the plant part is an inflorescence. In case the plant part of step a) is an inflorescence of a plant, a comparable inflorescence of a similar plant or the same plant may be used as a control. Increased pollen viability is preferably determined as an increase in viable pollen as may be established using an MTT assay as exemplified herein.

Preferably the increase in viable pollen is an increase in percentage of viable pollen, i.e. the amount of viable pollen over the total amount of pollen produced by a flower bud is increased. Preferably the increased percentage of viable pollen is an increase as compared to the percentage viable pollen produced by the control flower bud as defined herein. Preferably the increased viability is an increase of at least about 1%, 2%, 5%, 10%, 20%, 30%, 40%, 50%, 60%, 70%, 80%, 90% or 100%.

In addition or alternatively, the flower bud treated by the method of the invention has an increased number of open anthers preferably as compared to a control flower bud as defined herein. The increased number of open anthers may be an increase in the percentage of open anthers, i.e. the number of open anthers over the total number of anthers produced by the flower bud is increased. Preferably the increased percentage of open anthers is an increase as compared to the percentage open anthers produced by the control flower bud as defined herein. Preferably the increased number of open anthers is an increase of at least about 1%, 2%, 5%, 10%, 20%, 30%, 40%, 50%, 60%, 70%, 80%, 90% or 100%.

Alternatively or in addition, after exposure to an MRN-ATM pathway inhibitor as defined herein, preferably at least about 2%, 4%, 6%, 8%, 10%, 15%, 20%, 25%, 30%, 35%, 40%, 45%, 50%, 55%, 60%, 65%, 70%, 75%, 80%, 85%, 90%, 95% or 100% of the pollen produced by the tissue giving rise to plant pollen is viable. Preferably, less than about 95%, 90%, 85%, 80%, 75%, 70%, 65%, 60%, 55%, 50%, 45%, 40%, 35%, 30%, 25%, 20%, 15%, 10%, 8%, 6%, 4%, 2% or less than about 1% of the pollen produced by the tissue giving rise to plant pollen is inviable.

This increased pollen viability can be determined by an MTT assay, showing metabolic activity in the pollen. Preferably, at least 0.001%, 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, or 40% of the total amount of pollen from a tissue giving rise to plant pollen and that is treated by the method of the invention is viable as opposed to no more than 5%, 4%, 3%, 2%, 1%, 0.9%, 0.8%, 0.7%, 0.6%, 0.5%, 0.4%, 0.3%, 0.2%, 0.1%, 0.01%, 0.01%, 0.001% or 0.0001% of the total amount of pollen from a tissue giving rise to plant pollen and that is not being treated by the method of the invention.

Alternatively or in addition, after exposure of a tissue giving rise to plant pollen to an MRN-ATM pathway inhibitor as defined herein preferably at least about 2%, 4%, 6%, 8%, 10%, 15%, 20%, 25%, 30%, 35%, 40%, 45%, 50%, 55%, 60%, 65%, 70%, 75%, 80%, 85%, 90%, 95% or 100% of the formed anthers are open anthers. Preferably, less than about 95%, 90%, 85%, 80%, 75%, 70%, 65%, 60%, 55%, 50%, 45%, 40%, 35%, 30%, 25%, 20%, 15%, 10%, 8%, 6%, 4%, 2% or less than about 1% of the formed anthers remain closed.

Preferably, the increased amount of viable pollen and/or the increased number of open anthers results in an increased fertility of said plant. The method of the invention may therefore also be considered as a method to increase fertility of a plant.

After or simultaneously with treating the plant or plant part of step a) of the method of the invention, preferably the tissue giving rise to plant pollen, preferably an immature flower bud, preferably an inflorescence, with an MRN-ATM pathway inhibitor in the method of the invention, the method may further comprise the step of allowing the plant or plant part, to develop mature pollen. Such mature pollen or ripe pollen can subsequently be effectively used for pollination.

In case the plant part that has been treated with an MRN-ATM pathway inhibitor in the method of the invention has been cut off from the remainder of the plant, this treated plant part may subsequently be grafted on another plant part to further mature and develop into a whole plant. Preferably, the plant part treated by the method of the invention is an inflorescence that is subsequently grafted as a scion on a stock. Preferably, the plant part is a scion.

In a preferred embodiment the stock and MRN-ATM inhibitor treated scion are from the same or similar plant, preferably a plant being of the same genus, optionally of the species. Preferably, the stock and the scion are both from an offspring plant of the same parental cross. In another embodiment, the MRN-ATM inhibitor treated scion is from a different species as the plant providing the stock, however, the scion and stock are such that they are able to form a viable graft. The graft may subsequently further develop and the pollen may mature to the stage where they may be collected and/or used for subsequent pollination, preferably for cross-pollination or self-pollination.

Treating a plant part comprising an immature flower bud with a compound, as exemplified herein for mirin, and the subsequent grafting of that treated part as a scion on a rootstock, preferably a rootstock of the same genus, and subsequently successfully allowing the flower bud to fully mature as part of the grafted plant, may be considered a method that extends beyond the method of the present invention of increasing pollen viability. The skilled person understands that such method is suitable for use of treating such plant part with any compound affecting a process during early flower bud development, preferably during early pollen development, that is preferably used in small quantities, for instance because it is hazardous and/or toxic or because it is expensive or scarce. In particular, a method is provided for developing a fertile plant graft comprising the steps of:
(i) isolating a plant part comprising an immature flower bud, preferably an inflorescence;
(ii) contacting said plant part with a compound affecting a process in early flower bud development, preferably early pollen development;
(iii) grafting the plant part on a scion to form a viable graft; and
(iv) growing the graft to a fertile plant, preferably comprising fertile pollen.

The compound preferably is a compound affecting the meiotic process in plant gamete development, preferably pollen development. Preferably the plant is contacted with said compound during early maturation of the flower bud, preferably at least during microsporogenesis involving the pre-meiotic phase and the first meiotic division, and optionally also during the second meiotic division.

The method of the invention may further comprise a step of isolating the mature pollen. Preferably, the anthers comprising the mature viable pollen are open. Alternatively, closed anthers may be opened to expose the pollen. The viable pollen can be selected though a variety of methods, for example, but not limited to: sieving, centrifugation or fluorescence-activated flow sorting (FACS). Preferably the method of collecting viable pollen comprises a step of discarding the fraction of inviable pollen. Preferably the method of collecting viable pollen comprises a step of selecting the fraction of the pollen of a size comparable or equal to viable pollen, while discarding smaller and or larger pollen that may be inviable.

In a further embodiment, the viable mature pollen is used for pollinating the first seed plant and (self-pollination)/or a second plant (cross-pollination), optionally using isolated viable mature pollen. Pollination preferably results in the development of seed. The second plant is preferably a second seed plant. The second plant may be a plant that is of the same species as one of the parents of the first plant. Optionally, the second plant is a parent plant, i.e. the mother or the father plant, of the first seed plant. The second plant may be of the same species of the first plant. The pollination may be backcrossing. Optionally, the second plant is the same plant as the first plant. The pollination may be self-pollination. In case the first plant is a interspecific hybrid, the second plant may be a similar interspecific hybrid, i.e. having parent plants of the same species as the parent plants of the first plant, optionally having the same parent plants. The step of pollination using the viable pollen obtained by the method of the invention may be, but is not limited to crossing, back-crossing and self-pollination.

The second plant preferably provides the ovules for germination of the cross between the first and the second plant using the viable pollen of the first plant obtained by the method of the invention. Preferably, the second plant is the same plant as, or a sibling of, the first plant of the method of the invention.

The method may further comprise a step of seed development and optionally the production of a progeny plant.

Optionally, the method of the invention does not comprise an essentially biological step for the production of a plant.

In a further aspect, the invention provides for the use of an MRN-ATM pathway inhibitor as defined herein to increase pollen viability. Preferably said pollen is from an interspecific hybrid as defined herein. Preferably said MRN-ATM pathway inhibitor is an MRN complex inhibitor, more preferably an Mre11 inhibitor, even more preferably 2-Amino-5-[(4-hydroxyphenyl)methylene]-4(5H)-thiazolone or a derivative thereof.

In a further aspect, the invention provides for viable pollen obtained by the method of the invention as provided herein. Preferably the pollen has an improved viability. Preferably the pollen is mature pollen. Optionally, the pollen is isolated pollen, preferably isolated mature pollen. Preferably the pollen is pollen of an interspecific hybrid. Preferably, the pollen is of an interspecific hybrid, wherein (prior to performing the method of the invention) at least 10% of the pollen of the plant is inviable. Preferably the pollen is of an interspecific hybrid that is otherwise sterile. In other words, preferably the pollen of the invention is characterized in being viable, or having an increased viability, as opposed to pollen of a plant, preferably of an interspecific hybrid or part thereof, not being treated according to the method of the invention.

Optionally, the pollen of the invention has one or more genomic alterations that are due to or associated with a MRN-ATM pathway inhibitor, preferably that are due to or associated with treatment with an MRN complex inhibitor, more preferably treatment with an Mre11 inhibitor. Preferably, the viable pollen has one or more genomic alterations that are due to or associated with treatment with 2-Amino-5-[(4-hydroxyphenyl)methylene]-4(5H)-thiazolone or a derivative thereof.

Optionally, the viable pollen may be characterized in that the MRN-ATM pathway is inhibited, the MRN complex is inhibited and/or the Mre11 activity is inhibited. Optionally the viable pollen of the invention is characterized in that it contains 2-Amino-5-[(4-hydroxyphenyl)methylene]-4(5H)-thiazolone or traces of 2-Amino-5-[(4-hydroxyphenyl)methylene]-4(5H)-thiazolone.

In an aspect, the invention pertains to at least one of a plant, plant part, flower bud and an inflorescence comprising the pollen having improved viability as defined herein.

In an aspect, the invention pertains to a method for producing a first seed plant having an improved pollen viability, using the method steps as defined herein. Preferably, the method comprises the steps of:
a) providing a first seed plant or plant part comprising a tissue giving rise to plant pollen;
b) inhibiting the MRN-ATM pathway in at least part of the plant or part thereof of step a); and
c) allowing the contacted tissue to produce pollen, wherein the pollen shows improved viability.

In case a plant part is provided in step a), the method may further comprise a step of grafting the plant part onto a rootstock, as defined herein. Preferably, the provided plant part is a scion. Preferably, the tissue giving rise to plant pollen is part of an immature flower bud. Preferably, the immature flower bud is part of an inflorescence. Hence preferably, the plant part in step a) is an inflorescence.

In an aspect, the invention pertains to a seed plant comprising pollen that has an improved viability, wherein the seed plant is obtainable by the method of the invention as defined herein. The seed plant may be an interspecific hybrid and/or a graft. Optionally, a seed plant of the invention is identical to the first seed plant as defined herein, with the exception that the pollen has an improved viability.

Preferably, the seed plant comprising pollen having an improved viability is not, or is not exclusively, obtained by an essentially biological process. Preferably, the seed plant of the invention is obtained by a method comprising a technical step. Preferably the seed plant comprising pollen having an improved viability is manmade.

In an aspect, the invention concerns a seed and/or offspring produced using pollen having improved viability, wherein the pollen is obtainable by the method of the invention as defined herein. Said pollen is used as the father. The mother used for seed production is preferably at least one of the first and second plant as defined herein.

In a further aspect, provided is for the use of the pollen having improved viability of the invention in generating offspring by pollination. The pollination is preferably at least one of self-pollination and cross-pollination.

In a further aspect, the invention pertains to an agricultural carrier comprising a compound inhibiting the MRN-ATM pathway, preferably wherein the compound is 2-Amino-5-[(4-hydroxyphenyl)methylene]-4(5H)-thiazolone or a derivative thereof. Preferably the concentration of the compound in the agricultural carrier is about 5 μM, 4 μM, 3 μM, 2 μM, 1 μM, 900 nM, 800 nM, 700 nM, 600nM, 500 nM, 400 nM, 300 nM, 200 nM, 100 nM, 90 nM, 80 mM, 70 nM, 60 nM, 50 nM, 40 nM or 30 nM, and may be a concentration of at least about 1nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM or 20 nM. Preferably said concentration is in the range of between about 1 nM and 5 μM, between about 1 nM and 1 μM, between about 5 nM and 500 nM, between about 5 nM and 100 nM, between about 10 nM and 100 nM, between about 5 nM and 50 nM, between about 10 nM and 50 nM, or preferably between about 20 nM and 30 nM. A preferred agricultural carrier is selected from the group consisting of a plant growth medium, soil, fertilizer, plant-based oils and humectants. Preferably, the agricultural carrier is a plant growth medium.

A plant growth medium, or plant tissue culture growth medium, preferably supports plant viability and/or to promotes plant growth. A plant growth medium preferably promotes plant growth, preferably promotes the development of at least one of a plant root and a plant shoot. A plant growth medium preferably comprises at least (macro) nutrients. The plant growth medium may comprise at least one of nitrate, agar, sugars, vitamins and growth regulators. Preferred growth regulators are auxins, such as but not limited to IAA, and cytokinins, such as but not limited to kinetin. A preferred growth medium is Murashige and Skoog as described in in Murashige T. and Skoog F., supra.

### FIGURE LEGENDS

FIG. 1. An overview of the experimental setup, with the prepared anthers of the AC hybrid in a glass tube, containing 0.5 MS medium and the appropriated concentration of Mirin.

FIG. 2. An example of viable pollen (purple) mixed with non-viable pollen (brown).

FIG. 3. A range of different concentrations of Mirin applied to immature flower buds of a B. rapax B. oleracea hybrid. Viable pollen grains are visualized using a 2,5-diphenyl monotetrazolium bromide (MTT) assay, which stains viable pollen purple. A low amount 20 nM of Mirin resulted in a higher number of viable pollen grains. Higher concentrations resulted in larger but non-viable pollen.

FIG. 4. An open anther of a B. rapa x B. oleracea cross after application of 20 nM Mirin. Normally the F₁ of this cross does do not form open anthers.

### EXAMPLES

### Example 1. Application of Mirin Increases Fertility in Inter-Specific Hybrids

The inventors discovered that treatment of inflorescences of AC interspecific hybrids from a cross of Brassica oleracea and Brassica rapa with Mirin resulted in higher frequency of viable pollen grains as well as larger viable pollen grains. This is surprising as interspecific AC hybrids of B. oleracea and B. rapa are known to be sterile.

Interspecific hybrids (AC) between Brassica oleracea var. albograbra (C genome) as father and Brassica rapa var. albograbra (A genome) as mother were prepared by bud pollination and embryo rescue. B. oleracea and B. rapa parental lines were grown in the greenhouse at 20-22° C. in the greenhouse at 12 h day length. Once the plants flower, B. rapa was emasculated and pollinated with hand-pollen from B. oleracea.

At 5-9 days after cross-pollination, siliques that developed to the globular or heart-shaped embryo stages were dehisced and taken to in vitro conditions for embryo rescue. The obtained embryos were placed in Gamborg's B-5 media containing 1 mg·mL⁻¹ of 6-benzyloaminopuryne and 0.01 mg·mL⁻¹ of the auxin (indole-3-acetic acid). The embryos were then transferred to controlled conditions with a 12 h photoperiod and 15° C. day/10° C. night thermocycle. The best developed plants were transferred to the greenhouse and grown for further experimentation until inflorescence development.

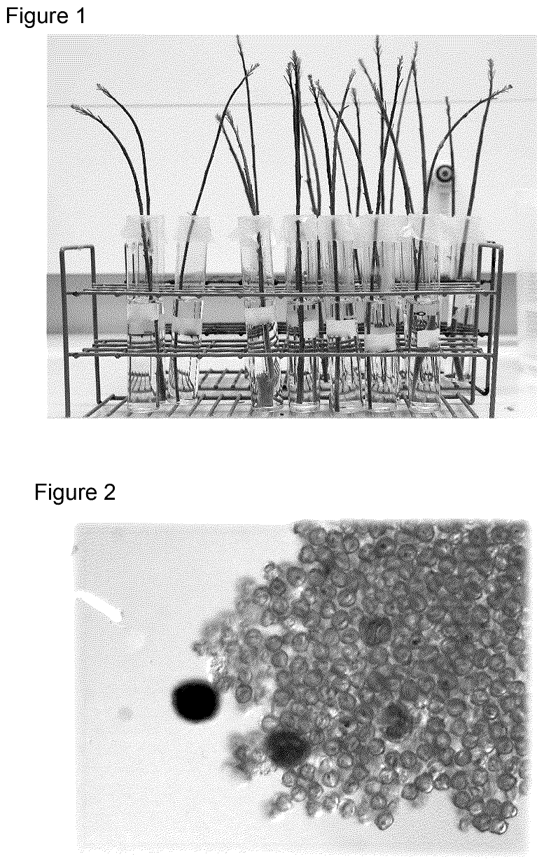

A concentration range of Mirin was prepared from 20 nM to 1 μM final concentration in 30ml 0.5 Muriashige skoog (MS) medium. This was added to a flat-bottom glass tube and sealed with parafilm. A small hole was made in the parafilm through which one inflorescence of the AC interspecific hybrid was placed. The inflorescence was prepared by removing all siliques, open flowers and mature flower buds, leaving only the very immature buds. An overview of the setup can be seen in FIG. 1. The resulting setup was placed under controlled conditions at 23° C./21° C. (day/night) and 16H/8H (light.dark) for 10 days. The pollen of opened flowers was collected and analyzed to determine the effect of Mirin on pollen viability.

Pollen viability was measured using a standardized 2,5-diphenyl monotetrazolium bromide (MTT) assay. This assay is based on mitochondrial activity, with viable cells turning a deep purple, while non-viable cells remain unstained. In short the assay uses a 1% MTT solution in 5% sucrose in demi H₂O, which is filtered to remove precipitation. The solution should be stored at 420 C. in the dark. A 50 μL drop of the MTT solution is dropped on a microslide and a small amount of pollen collected from opening the anthers with a needle is evenly distributed in the drop of MTT. After applying a glass cover slide and incubating at RT for 5-10 minutes, staining should be visible (FIG. 2). Using a microscope, the viable pollen can be scored.

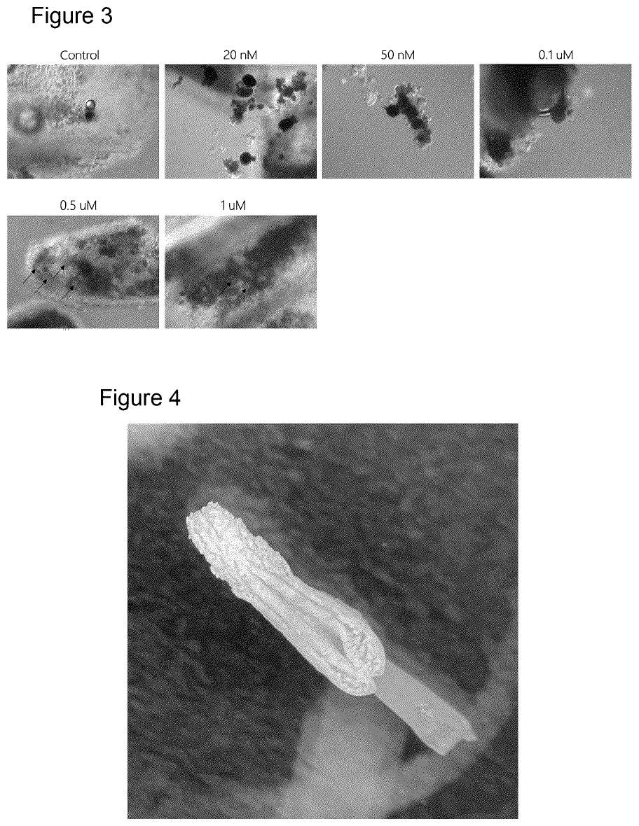

Observing pollen at different mirin concentrations showed that low concentrations of mirin (optimal at about 20nM) resulted in an increase of viable pollen (FIG. 3). In addition, this concentration of Mirin was found to result in and opening of anthers (FIG. 4). Concentrations above 0.5 μM were found to result in bigger but not viable pollen.

### Example 2. Allowing the Chemically Treated Inflorescences to Mature to Full Plants in the Greenhouse by Grafting

Brassica rapa rootstock cuttings were prepared by removing all leaves and axillary shoots of the stock leaving only the dominant shoot. The young immature chemically-treated inflorescences obtained from Example 1 were grafted onto these rootstock cuttings. For grafting, rootstocks were selected with a similar thickness as scion tops. Prior to grafting, the shoot tip or inflorescence of the stock was cut off using a scalpel blade (Swaan mortan #10). A vertical incision on the stock was made prior to grafting by moving the scalpel upwards to the cut end. A silicon clip big enough to hold the stock was used for grafting. A wedge or T was made in the scion, such that the edges were removed with the scalpel blade and the mid region of the scion was kept intact. The wedge shaped/T shaped scion was placed into the vertical incision of the stock and the silicon clip was moved gently but firmly such that it covered the graft junction. The grafted cuttings/plant was covered with a plastic cover such that it had ˜100% humidity for the first few days at least. After a week (7-9d) the grafts were healed. The silicon clips were removed from the graft junction. The plastic cover was removed and the chemically treated inflorescence were allowed to grow normally. This method allowed the flower buds to fully mature.

1. A method for improving the viability of plant pollen, comprising:
(a) obtaining a first seed plant or plant part thereof comprising a tissue giving rise to plant pollen;
(b) inhibiting the MRN-ATM pathway in at least part of the provided plant or plant part; and
(c) allowing the plant or plant part to produce pollen, wherein the pollen shows improved viability.

2. The method according to claim 1, wherein the inhibiting is by contacting the plant or plant part to 2-Amino-5-[(4-hydroxyphenyl)methylene]-4(5H)-thiazolone.

3. The method according to claim 1, wherein the improved viability of a plant pollen increases plant fertility.

4. The method according to claim 1, wherein the plant or plant part thereof is an interspecific hybrid or part thereof.

5. The method according to claim 1, wherein the plant part is a plant cutting, and wherein the cutting edge of the plant cutting is contacted in (b) with a compound that inhibits the MRN-ATM pathway.

6. The method according to claim 5, wherein the plant cutting is a cutting of an angiosperm seed plant and comprises an inflorescence, wherein the inflorescence comprises at least one immature flower bud.

7. The method according to claim 6, wherein the inflorescence does not comprise a mature flower bud.

8. The method according to claim 1, performed using a plant part that is a scion, and further comprising: (d) grafting the scion onto a stock.

9. The method according to claim 1, further comprising (d) allowing the pollen to mature.

10. The method according to claim 9, further comprising (e) isolating the mature pollen.

11. The method according claim 1, further comprising (d) self-pollinating the first plant or pollinating a second plant.

12. A plant growth medium, comprising a compound inhibiting the MRN-ATM pathway.

13. A viable pollen of a plant or plant part obtainable by a method according to claim 1.

14. A method for producing a first seed plant having improved pollen viability, comprising:
(a) providing a first seed plant or plant part thereof comprising a tissue giving rise to plant pollen;
(b) inhibiting the MRN-ATM pathway in at least part of the provided plant or plant part; and
(c) allowing the plant or plant part to produce pollen, wherein the pollen shows improved viability.

15. A seed plant, preferably an interspecific hybrid, comprising pollen having an improved viability, obtainable by the method of claim 14.

16. The seed plant according to claim 15, wherein the plant is an interspecific hybrid.
